(19) United States
(12) Patent Application Publication
(10) Pub. No.: US 2024/0130936 A1
(43) Pub. Date: Apr. 25, 2024

(54) WATER-IN-SILICONE COMPLEXION EMULSIONS

(71) Applicant: L V M H RECHERCHE, Saint-Jean-De-Braye (FR)

(72) Inventors: Emilie GOMBART, Orleans (FR); Isabelle Milcent, Orleans (FR); Marie Contier, Saint Pryve Saint Mesmin (FR); Myriam Chevalier, Boigny-Sur-Bionne (FR); Valérie Alard, Orleans (FR)

(73) Assignee: L V M H RECHERCHE, Saint-Jean-De-Braye (FR)

(21) Appl. No.: 18/535,261

(22) Filed: Dec. 11, 2023

Related U.S. Application Data
(63) Continuation of application No. 16/474,273, filed on Jun. 27, 2019.

(30) Foreign Application Priority Data
Dec. 28, 2016 (FR) 1663472

(51) Int. Cl. A61K 8/02; A61K 8/04; A61K 8/06; A61K 8/19; A61K 8/25; A61K 8/26; A61K 8/29; A61K 8/34; A61K 8/37; A61K 8/44; A61K 8/73; A61K 8/81; A61K 8/891; A61Q 1/02

(52) U.S. Cl. CPC A61K 8/0266; A61K 8/042; A61K 8/062; A61K 8/064; A61K 8/19; A61K 8/25; A61K 8/26; A61K 8/29; A61K 8/345; A61K 8/375; A61K 8/44; A61K 8/73; A61K 8/731; A61K 8/8152; A61K 8/891; A61Q 1/02; A61K 2800/43; A61K 2800/436

(57) ABSTRACT

The present invention relates in particular to a cosmetic composition for the care and/or make-up of keratinous materials, in particular the skin, comprising an aqueous phase and at least one combination with optical correction effect adapted to all skin complexions comprising at least one soft-focus effect filler, at least one composite pigment, and at least one reflective particle.

### FIELD OF THE INVENTION

The present invention relates to a cosmetic composition for the care and/or make-up of keratinous materials, in particular the skin, providing universal optical correction, i.e. adapted advantageously to all complexions.

### STATE OF THE ART

There are already in the prior art care and/or make-up products designed to mask skin relief imperfections, using soft-focus effect fillers, in particular silicone elastomers or silicon fillers. These products provide good blurring but with a generally matte finish, without any light effect and/or skin radiance.

In addition, make-up products, in particular foundation products (foundation, foundation powders) are generally available in a wide range of shades adapted to different complexions, particularly for light, medium and dark skin.

More recently, products have appeared in the form of loose powders (Translucent Loose Powder by Sephora) based on silica or compact powders (Poudre Compacte Radiance Perfectrice Universelle by YSL Beauté) based on synthetic mica, silicon filler and talc, offering an invisible finish and a luminous rendering, adapted to all skin types.

However, to meet the expectations of consumers who do not use powders (consumers with dry, rough skin or those who prefer more fluid and moisturizing galenicals), there is still a need to develop new cosmetic products apart from powder galenical, providing both an overall correction in transparency and a light effect, and which have a criterion of universality, i.e. are adapted to all skin complexions.

Patent application FR2858215 by L'Oreal discloses the use of a coloring agent (composite pigment) and a soft-focus effect filler in emulsions to provide color and hide skin defects while allowing the treated surface to retain a natural appearance. But these compositions do not have a light effect and are not suitable for all skin complexions (universality).

To the Applicant's knowledge, there are in the prior art no cosmetic compositions that comprise at least one aqueous phase and make it possible to obtain an overall corrective effect with a natural aspect and a luminous rendering, while being adapted to all complexions (universality criterion), i.e. conferring an optical correction that remains natural from light to dark skin.

Indeed, the foundations available on the market are generally offered in a wide range of shades, each shade being adapted to a given complexion. When a foundation adapted to a given complexion is applied to different complexions, the color difference increases and can become very large on opposite skin tones (much lighter or darker than the target shade). This color difference ΔE*₀₀ measured between the bare skin and the same skin to which said composition is applied may be greater than 20, or even greater than 40 for complexions very different from the color of the foundation.

### SUMMARY OF THE INVENTION

The present invention responds precisely to this need by proposing a cosmetic composition for the care and/or make-up of keratinous materials, in particular the skin, comprising an aqueous phase and at least one combination with optical correction effect adapted to all skin complexions, comprising:
(i) at least one soft-focus effect filler,
(ii) at least one composite pigment, and
(iii) at least one reflective particle.

According to the invention, “optical correction adapted to all skin complexions” means that the composition allows advantageously:
an immediate optical correction to be obtained: blurring and color but without matte effect, i.e. with a luminous skin finish;
that this immediate optical correction is universal, i.e. adapted to all skin complexions.

For the purposes of the present invention, “adapted to the complexion” means that the initial complexion (bare skin) is only slightly modified in terms of color after the composition of the invention has been applied to the same skin. Indeed, the color difference ΔE*₀₀ measured between the bare skin and the same skin on which the composition of the invention is applied is small, in particular less than 20 and even less than 10, and yet the skin is optically corrected.

Thus, the color difference between bare skin and the same skin on which the composition according to the invention is applied is notably reduced even for dark skin, whereas there were color differences ΔE*₀₀ which could be greater than 40 with the foundations known from the prior art, and this with nevertheless a real gain in terms of immediate perfection of the skin.

It is therefore possible to produce a single cosmetic composition adapted to the world market. In addition, providing a universal cosmetic composition will also greatly simplify the choice of shade for the consumer, compared to the current wide range of shades.

### DETAILED DESCRIPTION OF THE INVENTION

A first object of the invention relates to a cosmetic composition for the care and/or make-up of keratinous materials, in particular the skin, comprising an aqueous phase and at least one combination with optical effect adapted to all skin complexions, comprising:
(i) at least one soft-focus effect filler,
(ii) at least one composite pigment, and
(iii) at least one reflective particle.

Definition of the Universality Criterion and Measurement Method

For the purposes of the invention, a so-called “universal” composition or composition “with universal optical correction” refers to a composition whose optical correction obtained is adapted to all complexions, i.e. provides optical correction that remains natural from light to dark skin.

The universality criterion represents the ability of a formula to suit all skin complexions.

The universality criterion of a composition according to the invention can be observed directly with the naked eye: the composition is said to be universal if there is no notable difference in color between the bare skin and the same skin on which a composition according to the invention is applied. A color matching system or color card can also be used.

Skilled persons will be able to define other methods demonstrating a color difference in skin before/after application of a composition.

According to a particular embodiment, the universality criterion of a composition according to the invention is measured according to the in vitro measurement protocol developed and described as follows:

The formula is spread on a “contrast card” type support with light beige to dark brown colored areas (5 distinct colored areas). Spreading is carried out by means of an automatic device (REMATIQUE) and a BIRD type bar, adjusted to a 30-μm air gap.

The film is dried in a controlled way for 2 h at 40° C.

After drying, a support adapted to measurements on fragile film is placed on the film. Measurements are made using a MA98 multi-angle spectrophotometer (XRITE) placed on the support.

The measurement conditions are as follows: direct geometry: light source at 45° from vertical, measurement angle 0° from vertical; source D65; observer 10°.

Measurements are made on the different colored areas of the support, one part bare and the other covered with a cosmetic product. The color difference ΔE*₀₀ is then calculated between the values obtained with and without a cosmetic product for the same background color. ΔE*₀₀ is as defined by the CIE (ISO/CIE Standard 11664-6:2014)

The lower the ΔE*₀₀ is, the lower the impact of the cosmetic product on the initial background color. To achieve the expected level of universality for a cosmetic product, this color difference must be small, in particular less than 20, regardless of the color of the background to which it has been applied.

Preferably, the composition according to the invention is characterized by a color difference ΔE*₀₀ measured between the bare skin and the same skin on which said composition is applied, less than 20, preferentially less than 10, regardless of the skin complexion for which the color difference ΔE*₀₀ is measured.

Aqueous Phase

The aqueous phase of a composition according to the invention consists of water and optionally a water-soluble solvent.

According to the invention, “water-soluble solvent” means a compound that is liquid at room temperature and miscible with water (miscibility in water greater than 50 wt % at 25° C. and atmospheric pressure). Particular mention may be made of:
lower C₁-C₅ monoalcohols such as ethanol, isopropanol and mixtures thereof;
C₂-C₈ glycols such as ethylene glycol, propylene glycol, 1,3-butylene glycol, dipropylene glycol, and mixtures thereof;
C₂-C₃₂ polyols such as polyglycerols, polyethylene glycols, and mixtures thereof, and mixtures thereof.

According to one embodiment, the aqueous phase and the combination with optical correction effect of the invention are present within the same composition.

The aqueous phase may be present in the composition of the invention in a content ranging from 1% to 99% based on the total weight of said composition.

According to a particular embodiment, the 3 compounds of the combination with optical correction effect are dispersed in the same phase. According to another particular embodiment, the 3 compounds of the combination with optical correction effect are dispersed in distinct phases.

According to another embodiment, the aqueous phase and the combination with optical correction effect of the invention are packaged in two distinct sets. The combination of optical correction effect is brought into contact with the aqueous phase before application to the skin.

Another object of the invention therefore relates to a cosmetic care kit for the care and/or make-up of keratinous materials, in particular the skin, comprising
(a) a first composition comprising an aqueous phase and
(b) at least one soft-focus effect filler, at least one composite pigment, and at least one reflective particle, packaged in one or more compositions distinct from the first composition, for the extemporaneous preparation of a cosmetic composition as defined according to the invention.

Soft-focus effect fillers, composite pigments and reflective particles are as defined below.

The invention also relates to a process for the extemporaneous preparation of a cosmetic composition according to the invention, characterized in that it comprises the extemporaneous mixing of a first composition comprising an aqueous phase with at least one soft-focus effect filler, at least one composite pigment, and at least one reflecting particle packaged in one or more compositions distinct from the first composition.

The first composition comprising the aqueous phase and the composition(s) comprising soft-focus effect fillers, composite pigments, and reflective particles can be packaged in single-dose containers (e.g. capsules) that the consumer can mix just before application of the resulting composition.

Soft-Focus Effect Fillers

The composition according to the invention comprises at least one soft-focus effect filler.

For the purposes of the invention, “soft-focus effect” means an effect that camouflages the microreliefs of the skin and thus reduces by optical correction imperfections of relief and/or color, such as wrinkles, fine lines, pores and spots.

Hereinbelow, the terms “filler with soft-focus effect” and “soft-focus effect filler” are used interchangeably.

For the purposes of the invention, “fillers” means colorless or white particles of a mineral or organic, natural or synthetic nature, which are in insoluble form and dispersed in the medium of the composition. The soft-focus effect fillers used according to the invention are preferably non-composite fillers, distinct from the composite pigments described below.

By “soft-focus effect fillers” according to the invention, advantageous mention may be made of the fillers selected from the group consisting of:
cellulose powders, cellulose beads,
microcrystalline cellulose powders
silica and silicate powders, amorphous silica microspheres, silica microbeads
poly(methyl methacrylate) (PMMA) powders
boron nitride powders
cross-linked elastomeric organopolysiloxane powders optionally coated with silicone resin
hydrophobic silica aerogel powders,
nylon powders
starch powders
powders of vegetable origin, such as rice powders, cotton powders, silk powders and mixtures thereof.

According to a preferred embodiment of the invention, the cosmetic composition comprises at least one soft-focus effect filler selected from the group consisting of: cellulose powders, microcrystalline cellulose powders, starch powders, silica or silicate powders, poly(methyl methacrylate) (PMMA) powders, boron nitride powders, cross-linked elastomeric organopolysiloxane powders, hydrophobic silica aerogel powders, powders of vegetable origin and mixtures thereof, more preferably cellulose powders, poly(methyl methacrylate) (PMMA) powders, cross-linked elastomeric organopolysiloxane powders, hydrophobic silica aerogel powders and mixtures thereof, and mixtures thereof.

According to a particular preferred embodiment, the combination of cellulose powders and cross-linked elastomeric organopolysiloxane powders will be used as soft-focus effect fillers.

According to another particular preferred embodiment, the combination of poly(methyl methacrylate) (PMMA) and hydrophobic silica aerogel powders will be used as soft-focus effect fillers.

According to a particular embodiment, the soft-focus effect fillers used according to the invention are selected from:
cellulose powders with irregular contours, such as those marketed by Daito Kasei under the name Cellulobeads USF;
microcrystalline cellulose powders, such as those with the INCI name “Microcrystalline Cellulose”, marketed by Rettenmaier under the name Vivapur CS 4 FM;
silica powders with the INCI name “silica” marketed by JGC under the name Spherica BA1;
cross-linked elastomeric organopolysiloxane powders coated with silicone resin, in particular silsesquioxane resin, such as powders with the INCI names “Vinyl dimethicone/methicone silsesquioxane crosspolymer PEG-7 glyceryl cocoate, Polyquaternium-7 and methylsilanol tri-PEG-8 glyceryl cocoate” marketed by Miyoshi Kasei under the name MW-SRP-100;
PMMA powders such as the powder with the INCI name “methyl methacrylate crosspolymer” from Sunjin under the name SUNPMMA-X or MAKIBEADS 150 from Daito Kasei;
hydrophobic silica aerogel particles with the INCI name Silica silylates marketed by Dow Corning under the name Dow Corning VM-2270 Aerogel fine particles;
and mixtures thereof.

Preferably, the following soft-focus effect fillers will be used:
cellulose powders with irregular contours, such as those marketed by Daito Kasei under the name Cellulobeads USF;
cross-linked elastomeric organopolysiloxane powders coated with silicone resin, in particular silsesquioxane resin, such as powders with the INCI names “Vinyl dimethicone/methicone silsesquioxane crosspolymer PEG-7 glyceryl cocoate, Polyquaternium-7 and methylsilanol tri-PEG-8 glyceryl cocoate” marketed by Miyoshi Kasei under the name MW-SRP-100;
PMMA powders such as the powder with the INCI name “methyl methacrylate crosspolymer” from Sunjin under the name SUNPMMA-X or MAKIBEADS 150 from Daito Kasei;
hydrophobic silica aerogel particles with the INCI name Silica silylates marketed by Dow Corning under the name Dow Corning VM-2270 Aerogel fine particles;
and mixtures thereof, optionally in pairs.

These soft-focus effect fillers can advantageously be surface treated with a hydrophilic or lipophilic organic agent to facilitate their incorporation into either phase of the composition. These organic agents can be selected from amino acids; waxes; fatty acids, fatty alcohols and derivatives thereof; anionic surfactants; lecithins; sodium, potassium, magnesium, iron, titanium, zinc or aluminum salts of fatty acids; metal alkoxides; polysaccharides, cellulose and derivatives thereof; polyethylene; (meth)acrylic polymers, for example poly(raethyl rnethacylate)s; polymers and copolymers containing acrylate units: silicon compounds, for example silicones, polydimethylsiloxanes, alkoxysilanes, alkyisilanes, siloxy-silicates; fluorinated organic compounds, for example perfluoroalkyi ethers; fluoro-silicon compounds.

According to a particular embodiment of the invention, the soft-focus effect fillers comprise or consist of spherical particles, spherical particles with regular or irregular contours, or platelet particles, having a median volume size D50 less than or equal to 25 μm, notably less than or equal to 15 μm, in particular less than or equal to 10 μm.

“Median volume size” means the dimension given by the statistical particle size distribution to half of the population, known as D50 measured with the Malvern-Mastersizer Laser

Particle Size Analyzer.

A composition according to the invention may comprise from 0.1 to 40 wt %, preferably from 1 to 10 wt % of soft-focus effect filler(s), based on the total weight of said composition.

The compositions according to the invention may also contain additional conventional fillers, it being understood that skilled persons will take care not to choose fillers whose nature or quantity in the composition would impact the soft-focus effect provided by the soft-focus effect fillers. Particular examples of additional fillers distinct from the soft-focus effect fillers mentioned above include talcs, natural and synthetic micas, sericites, borosilicates, and mixtures thereof.

According to a particular embodiment of the invention, the soft-focus effect fillers are characterized by a haze index ranging from 40 to 100, preferably from 60 to 100, preferably from 70 to 100, measured for example according to the protocol described below.

Skilled persons will be able, on the basis of their knowledge, to define other methods enabling them to define this haze index.

According to a particular embodiment, they will use the measurement method described below.

Definition of the Haze Index and Measurement Method

The haze index measurement was developed according to ASTM D1003. The haze index is defined here as the proportion of incident radiation that, after passing through the sample, is scattered in relation to the total transmitted radiation.

The efficiency of the fillers to blur imperfections is evaluated in vitro by spectrophotometric transmission measurements of a thin film of formula. The raw materials to be evaluated are first incorporated at 2% in a cosmetic formula called “simplex” containing an aqueous phase. The formula is spread on a transparent support. Spreading is carried out by means of an automatic device (REMATIQUE) and a BIRD type bar, adjusted to a 30-μm air gap.

The film is dried in a controlled way for 2 h at 40° C.

Total transmission (Tt) and diffuse transmission (Td) measurements are performed with an integrating sphere spectrophotometer (COLOR 15, GRETA MACBETH) after calibration with a white reference tile and a light trap.

The measurement range is from 400 nm to 700 nm. The results are included in this range. The Haze value is calculated according to the following formula in accordance with the above standard:

H=Haze=100×(Td/Tt)

The Haze obtained can be comprised between 0 and 100.

Two cosmetic formulas called simplex are used as a basis for evaluating each raw material:
A direct O/W emulsion for the evaluation of all raw materials with a preferentially hydrophilic character;
A reverse W/O emulsion for the evaluation of all raw materials with a preferentially lipophilic character.

The contents in the simplex formulas below are expressed as wt % of raw material based on the total weight of the composition.

O/W simplex

| | |
|---|---|
| WATER | qs 100 |
| GLYCOLS | 12.0% |
| NON-VOLATILE HYDROCARBON OIL | 3.0% |
| FILM-FORMING AGENTS | 10.5% |
| GELLING AGENTS | 0.9% |
| PRESERVATIVE | qs |

W/O simplex

| | |
|---|---|
| WATER | qs 100 |
| VOLATILE SILICON OIL | 21.9% |
| GLYCOLS | 11.0% |
| GLYCOL ESTERS | 6.5% |
| GELLING AGENTS | 4.8% |
| FILM-FORMING AGENTS | 2.0% |
| SURFACTANT | 5.0% |
| PRESERVATIVE | qs |

The soft-focus effect fillers particularly preferred in the present invention are illustrated in Tables 1 and 2 below:

TABLE 1
Hydrophilic raw materials in O/W simplex

| | | | Haze index |
|---|---|---|---|
| | | | measured |
| Raw material RM | | | according to the |
| commercial designation | INCI name | Shape | above protocol |
| Talc | Talc | platelet | 37 |
| Talc Powder EX15 | | | |
| (comparative - non-soft- | | | |
| focus effect filler) | | | |
| USF Cellulobeads from | cellulose | Non-spherical | 95 |
| Daito Kasei | | (potato-shaped) | |
| MW-SRP-100 from | Vinyl | sphere | 87 |
| Miyoshi | dimethicone/methicone | | |
| | silsesquioxane | | |
| | crosspolymer/PEG-7 | | |
| | glyceryl cocoate, | | |
| | polyquaternium-7 and | | |
| | methylsilanol tri-PEG-8 | | |
| | glyceryl cocoate | | |
| Sunpmma X from Sunjin | Methyl methacrylate | Hemi-spherical | 94 |
| Chemicals | crosspolymer | | |
| Spherica BA1 from JGC | silica | spherical | 68 |
| Vivapur CS 4 FM from | Microcrystalline | fibers | 88 |
| Rettenmaier | Cellulose | | |
| REMYTEC F-I | Oryza Sativa Starch | Non-spherical, | 75 |
| | | angular particles | |
| SILK COTTON PW | Cellulose | Non-spherical | 87 |
| | | (potato-shaped) | |

TABLE 2
Lipophilic raw materials in W/O simplex

| Raw material | | | Haze index |
|---|---|---|---|
| RM | | | measured |
| commercial | | | according to the |
| designation | INCI name | Shape | above protocol |
| Dow Corning | silica silylate | Non-spherical | 96 |
| VM-2270 | | (potato-shaped) | |
| Aerogel | | | |
| fine particles | | | |
| from Dow | | | |
| Corning | | | |
| Makibeads | Methyl methacrylate | sphere | 72 |
| 150 from Daito | crosspolymer | | |
| Kasei | | | |

The soft-focus effect fillers according to the invention therefore have a haze index ranging from 40 to 100, preferably from 60 to 100, more preferably from 70 to 100 according to the protocol described above.

Composite Pigments

For the purposes of the invention, “composite pigment” means in particular a pigment comprising a substrate and inorganic and/or organic pigment(s) which are embedded in said substrate.

According to a particular embodiment, said composite pigment is surface treated and the coating obtained can be continuous or discontinuous.

The substrate may be a matrix or a core comprising at least one inorganic material and/or at least one organic material.

The inorganic material of the substrate of said composite pigment can be selected from the group consisting of: mica, synthetic mica, talc, sericite, boron nitride, glass or borosilicate flakes, calcium carbonate, barium sulfate, titanium oxide, hydroxyapatite, silica, silicate, zinc oxide, magnesium sulfate, magnesium carbonate, magnesium trisilicate, aluminum oxide, aluminum silicate, alumina, calcium silicate, calcium phosphate, magnesium oxide, bismuth oxychloride, kaolin, mineral clay, synthetic clay, and mixtures thereof.

In a preferred embodiment, the substrate of the composite pigment is composed of silica, borosilicate, alumina, or mixtures thereof.

The organic material of the substrate of the composite pigment can be selected from the group consisting of: poly(methyl methacrylate)s (PMMAs), polyamides, silicones, polyurethanes, polyethylenes, polypropylenes, polystyrenes, polysaccharides, cellulose, polyvinyl alcohols, polyvinyl resins, waxes, and mixtures thereof.

In a preferred embodiment, the substrate of the composite pigment is composed of silicone, polyurethane, poly(methyl methacrylate)s (PMMAs), cellulose, and mixtures thereof.

In a particularly preferred embodiment, the composite pigment comprises a silica-based substrate.

In another preferred embodiment, the composite pigment comprises a poly(methyl methacrylate) (PMMA)-based substrate.

The inorganic pigments of said composite pigment can be selected in particular from titanium or zirconium oxides or dioxides; zinc, iron or chromium oxides; ferric blue; manganese violet; ultramarine blue and chromium hydrate and mixtures thereof.

According to a preferred embodiment, the inorganic pigments of said composite pigment are selected from titanium oxides or dioxide, iron oxides and mixtures thereof.

The organic pigments of said composite pigment can be selected from carbon black, D&C type pigments, and lacquers based in particular on cochineal carmine, barium, strontium, calcium, aluminum and mixtures thereof.

According to a preferred embodiment, the organic pigments of said composite pigment are selected from D&C type pigments, lacquers and mixtures thereof.

These composite pigments result from the encapsulation and/or embedding of mineral or organic pigments in or on substrates or matrices of different natures in order to bring new optical properties to the pigments: in particular to bring a lot of transparency while bringing soft-focus properties.

The color obtained in mass with these composite pigments has the same hue, luminance and saturation as with conventional mineral pigments, with the same weight of raw material, but the coverage can be reduced by more than 50%, thus conferring a real gain in the natural effect of the products containing them after application to the skin.

Skilled persons will be able to choose the appropriate composite pigments according to the desired effect: a greater or lesser transparency and/or perfective effect depending on the matrix of said composite pigment, a different compatibility depending on the matrix of said composite pigment to promote its incorporation in the aqueous, oil or silicon phase.

The composite pigments according to the invention are in particular selected from:
spherical silica particles in which inorganic pigments, in particular titanium oxides and dioxide, iron oxides and mixtures thereof, are embedded; such as the SPHERICA HC family from JGC;
silicone particles in which inorganic pigments, in particular titanium oxides and dioxide, iron oxides and mixtures thereof, are embedded;
polyurethane particles in which inorganic pigments, in particular titanium oxides and dioxide, iron oxides and mixtures thereof, are embedded;
poly(meth)acrylate particles, in particular poly(methyl methacrylate) (PMMA) particles in which inorganic pigments, in particular titanium oxides and dioxide, iron oxides and mixtures thereof, are embedded; advantageous mention may be made of hemispherical poly(methyl methacrylate) (PMMA) particles coated with inorganic pigments of the 3D-Tech family marketed by DAITO;
spherical cellulose particles in which pigments, particularly inorganic ones, such as the Cellulobeads D10 family (<15 μm), are embedded;
alumina particles on which inorganic and/or organic pigments are embedded, and advantageously surface treated with silicon compounds of type Triethoxycaprylylsilane for better water resistance and better skin adhesion;
examples include the composite pigments of the Covalumine AS family marketed by Sensient,
and mixtures thereof.

According to a preferred embodiment, the composition of the invention comprises at least one composite pigment selected from spherical silica particles in which inorganic pigments, in particular titanium oxides and dioxide, iron oxides and mixtures thereof, are embedded; such as the SPHERICA HC family from JGC.

According to another preferred embodiment, the composition of the invention comprises at least one composite pigment selected from hemispherical poly(methyl methacrylate) (PMMA) particles coated with inorganic pigments of the 3D-Tech family marketed by DAITO.

The composite pigments according to the invention will be present in a content ranging from 1 to 20 wt %, preferably from 1 to 10 wt %, based on the total weight of the composition. These wt % represent the weight of raw material relative to the total weight of the composition.

The cosmetic composition according to the invention may also contain inorganic and/or organic pigments distinct from the composite pigments, provided that they do not impact the transparent color effect provided by the above-mentioned composite pigments.

The content of inorganic and/or organic pigments distinct from the composite pigments may range from 0 to 5 wt %, preferably from 0 to 2 wt %, based on the total weight of said composition.

According to a particular and preferred embodiment, the composition is free of inorganic and/or organic pigments distinct from the composite pigments.

According to a particular embodiment of the invention, the composite pigments are characterized by an opacity less than or equal to 75, preferably less than or equal to 60 or less than or equal to 50, and more preferably less than or equal to 20, measured in particular according to the protocol developed and described below.

Skilled persons have the knowledge to define other methods allowing them to measure the opacity of a sample.

According to a particular embodiment, they will use the measurement method described below.

Definition of Opacity and Measurement Method

The opacity of the sample is a ratio between the luminances Y of the sample measured on a black and white background.

The raw material to be studied is first dispersed at 2% in a transparent film-forming base.

This transparent film-forming base is of conventional composition consisting essentially of a film-forming polymer such as nitrocellulose, at least one plasticizer and at least one rheology agent solubilized and/or dispersed in a solvent or a solvent mixture (butyl acetate and ethyl acetate).

The formula is spread on a “contrast card” type support with at least one black area and one white area. Spreading is carried out by means of an automatic device (REMATIQUE) and a BIRD type bar, adjusted to a 30-μm air gap.

The film is dried in a controlled way for 2 h at 40° C.

After drying, a support adapted to measurements on fragile film is placed on the card. Measurements are made using a MA98 multi-angle spectrophotometer (XRITE) placed on the support.

The measurement conditions are as follows: direct geometry: light source at 45° from vertical, measurement angle 0° from vertical; source D65; observer 10°.

Two measurements are made for each sample: One measurement Y_{fb} on the black area of the card covered with cosmetic product, another measurement Y_{fw} on the white area of the card covered with cosmetic product. The ratio of the luminances Y obtained is then calculated:

O=Opacity=Y_{fb}/Y_{fw}

The opacity can be comprised between 0 and 100.

The composite pigments particularly preferred in the present invention with an opacity less than or equal to 75, preferably less than or equal to 60 or less than or equal to 50, are illustrated in Table 3 below:

TABLE 3

| | | | Opacity (or |
|---|---|---|---|
| | | | transparency) |
| Raw material RM | | | measured |
| commercial | | | according to the |
| designation | INCI name | Shape | above protocol |
| C 338021 SUN PURO | Iron oxide CI 77491 | Non-spherical | 98.2 |
| RED IRON OXIDE | | | |
| (comparative - non- | | | |
| composite pigment) | | | |
| SPHERICA HC RW 30 | Red iron oxide | spherical | 14.2 |
| from JGC | CI 77491 and silica | | |
| 3DTECH PW OTS | Methyl Methacrylate | Hemi-spherical | 57.8 |
| IOR-30 from Daito | Crosspolymer and iron | | |
| | oxide CI 77491 and | | |
| | Triethoxycaprylylsilane | | |
| Cellulobeads D-10 (R- | Cellulose and Iron Oxide | spherical | 6.2 |
| 33P) from Daito | CI 77491 | | |
| Covalumine Sonoma | Alumina + Iron oxide CI | platelets | 56.1 |
| Red AS from Sensient | 77491 and | | |
| | Triethoxycaprylylsilane | | |

Reflective Particles

According to the invention, “reflective particles” means multilayer structures comprising a substrate coated with inorganic and/or organic pigments, whose size, structure and physical and chemical nature of the layers, as well as their surface state, reflect incident light and create highlights on the skin after application of said composition containing them.

The reflective particles can have a variety of shapes, including platelet-shaped or globular, preferably platelet-shaped.

The term “platelet” refers to particles whose ratio of the largest dimension to thickness is greater than or equal to 5, preferably greater than or equal to 10, the thickness being generally comprised between 0.1 μm and 5 μm.

The reflective particles can be selected in particular from the group consisting of:
particles with a natural or synthetic substrate coated at least partially with at least one layer of at least one metal compound, in particular at least one metal oxide;
the particles formed by a stack of at least two layers of materials with different refractive indices, at least one of these layers which may be a polymer,
and mixtures thereof.

According to a particular embodiment, the reflective particles have a substrate selected from borosilicate, alumina, natural or synthetic mica, aluminum, silica, bismuth oxychloride, and mixtures thereof.

More preferably, reflective particles with a borosilicate, natural or synthetic mica or alumina substrate will be used.

Inorganic and/or organic pigments are such as those described above.

According to a particular embodiment, the reflective particle consists of multilayer platelet structures comprising a borosilicate, alumina, natural or synthetic mica substrate, or mixtures thereof, coated with inorganic and/or organic pigments, preferably an alumina or synthetic mica substrate coated with inorganic and/or organic pigments

By way of examples of reflective particles that can be used in the composition of the invention, particular mention may be made of:
particles comprising a borosilicate substrate coated with a metal compound, in particular titanium dioxide CI77891, such as those marketed under the name Reflecks Dimension Luminous White by BASF;
particles comprising a synthetic mica substrate coated with a metal compound, in particular titanium dioxide CI77891, such as those marketed under the name Helios R10R by TOPY; particles comprising a natural mica substrate coated with a metal compound, in particular titanium dioxide CI77891, such as those marketed under the name Flamenco Summit Aqua A80H by BASF;
particles comprising a synthetic mica substrate coated with a metal compound, in particular titanium dioxide CI77891, such as those marketed under the name Timiron Halo White by MERCK;
particles comprising a synthetic mica substrate coated with a metal compound, in particular CI77891 and CI77491, such as those marketed under the name Syncrystal Soft Peach by Eckart;
particles comprising an alumina substrate coated with titanium dioxide under the name SpectraFlex Focus White from SUN CHEMICAL,
and mixtures thereof.

Preferably, particles comprising an alumina substrate coated with titanium dioxide under the name SpectraFlex Focus White from SUN CHEMICAL, particles comprising a synthetic mica substrate coated with a metal compound, in particular titanium dioxide CI77891, such as those marketed under the name Timiron Halo White by MERCK; and mixtures thereof.

According to a preferred embodiment, a combination of particles comprising an alumina substrate coated with titanium dioxide under the name SpectraFlex Focus White from SUN CHEMICAL, and a synthetic mica substrate coated with titanium dioxide under the name Timiron Halo White from MERCK, will be used.

The reflective particles according to the invention generally have a D90 less than or equal to 40 μm, preferably less than or equal to 25 μm, more preferably less than or equal to 20 μm.

The reflective particles will be present in the composition of the invention in a content ranging from 0.05 to 10 wt % based on the total weight of the composition, preferably from 0.1 to 5 wt % based on the total weight of the composition.

According to a particular embodiment, the reflective particles used in the cosmetic compositions of the invention are characterized by a diffuse brightness measurement ranging from 1200 to 3000, in particular from 1300 to 2500, measured for example according to the protocol developed and described below.

Skilled persons will be able to define other measurement methods allowing them to measure the diffuse brightness of reflective particles suitable for the invention.

According to a particular embodiment, they will use the method described below.

Definition of the Light Effect and Measurement Method

According to the state of the art, the light effect describes the way a product shines. Brightness, which corresponds to the specular reflection of a surface, is perceived by the observer as a luminous spot. At equivalent intensity, this spot may be more or less large; the brightness is then perceived as intense or diffuse.

The raw materials to be evaluated are first incorporated at 2% in a simplex cosmetic formula of the O/W emulsion type.

O/W simplex

| | |
|---|---|
| WATER | qs 100 |
| GLYCOLS | 12.0% |
| NON-VOLATILE HYDROCARBON OIL | 3.0% |
| FILM-FORMING AGENTS | 10.5% |
| GELLING AGENTS | 0.9% |
| PRESERVATIVE | qs |

The formula is spread on a “contrast card” type support with at least one area of color. Spreading is carried out by means of an automatic device (REMATIQUE) and a BIRD type bar, adjusted to a 30-μm air gap.

The film is dried in a controlled way for 2 h at 40° C.

The measurements are carried out using a contactless image acquisition and processing device (Samba, BOSSA NOVA TECH) consisting of a light source, a polarizing camera, a curved support and processing software. The system allows the angular distribution of the light energy reflected by the sample to be studied. The curve of the specular light energy is thus plotted as a function of the measurement angle, it is bell-shaped. The area under the curve is calculated, the value of which allows the light effect of a cosmetic product to be evaluated.

The reflective particle pigments particularly preferred in the present invention are illustrated in Table 4 below:

TABLE 4

| | | | | Light effect |
|---|---|---|---|---|
| | | | | (area under |
| | | | | the specular |
| | | | | reflection |
| | | | | curve) |
| | | | | measured |
| | | | | according to |
| | | | | the above- |
| Raw material | | | | mentioned |
| RM | | | | protocol (AU = |
| commercial | | | | arbitrary |
| designation | INCI name | Shape | Size D90 | units) |
| Simplex | | | | 919 |
| formula | | | | |
| without | | | | |
| mother-of- | | | | |
| pearl | | | | |
| Reflecks | Calcium Sodium Borosilicate | platelets | 39 μm | 1368 |
| Dimension | and Titanium Oxide CI 77891 | | | |
| Luminous | and Tin Oxide | | | |
| White G130M | | | | |
| from BASF | | | | |
| Timiron Halo | Synthetic Fluorphlogopite and | platelets | 15 μm | 2295 |
| White from | Titanium Dioxide CI 77891 | | | |
| MERCK | and Tin Dioxide | | | |
| ECKART | Synthetic Fluorphlogopite and | platelets | 15 μm | 2112 |
| Syncrystal | Titanium Dioxide CI 77891 | | | |
| Soft Peach | and Tin Dioxide and Iron | | | |
| | Oxide CI 77491 and Tin | | | |
| | Dioxide | | | |
| Flamenco | Mica and Titanium Dioxide CI | platelets | 34 μm | 1920 |
| Summit Aqua | 77891 and Silica and Tin | | | |
| A80H from | Oxide | | | |
| BASF | | | | |
| Helios R10R | Synthetic Fluorphlogopite and | platelets | 25 μm | 1283 |
| from TOPY | Titanium Dioxide CI 77891 | | | |
| | and Tin Dioxide | | | |
| SpectraFlex | Alumina Titanium Dioxide | platelets | 18 μm | 1673 |
| Focus White | | | | |
| from SUN | | | | |
| CHEMICAL | | | | |
| Sunshine Fine | Synthetic Fluorphlogopite and | platelets | 20 μm | 2383 |
| White from | Titanium Dioxide | | | |
| SUN | | | | |
| CHEMICAL | | | | |

All the combinations with universal optical correction effect comprising at least one soft-focus effect filler, one composite pigment and one reflective particle, among the lists of compounds described above in the description and in particular those illustrated respectively in Tables 1 to 4 described above, are part of the present invention. Particular mention may be made of combinations comprising at least three compounds or more, in particular four compounds or five compounds, among those listed above, including at least one soft-focus effect filler, one composite pigment and one reflective particle. According to a particular embodiment and as illustrated in the examples described below, at least two soft-focus effect fillers, a composite pigment and two reflective particles may be used in the cosmetic composition of the invention.

In particular, the combinations described in Table 5 below can be used:

TABLE 5

| | | | | | | |
|---|---|---|---|---|---|---|
| | | | Spherical silica | | Spherical | Alumina |
| | | | particles in | Poly(meth)acrylate | cellulose | particles on |
| | | | which | particles in | particles in which | which inorganic |
| | | | inorganic | which inorganic | inorganic | and/or organic |
| | Soft-focus | Composite | pigments are | pigments are | pigments are | pigments are |
| Reflective particles | effect fillers | pigments | embedded | embedded | embedded | embedded |

| | | | | | |
|---|---|---|---|---|---|
| Particles comprising a | Cellulose powders | x | x | x | x |
| borosilicate substrate coated | Silica powders | x | x | x | x |
| with a metal compound, in | Poly(methyl | x | x | x | x |
| particular titanium dioxide | methacrylate) | | | | |
| | (PMMA) powders | | | | |
| | Elastomeric | x | x | x | x |
| | organopolysiloxane | | | | |
| | powders | | | | |
| | Hydrophobic silica | x | x | x | x |
| | aerogel powders | | | | |
| | Cellulose powders | x | x | x | x |
| | and elastomeric | | | | |
| | organopolysiloxane | | | | |
| | powders | | | | |
| | Poly(methyl | x | x | x | x |
| | methacrylate) | | | | |
| | (PMMA) and | | | | |
| | hydrophobic silica | | | | |
| | aerogel powders | | | | |
| Particles comprising a | Cellulose powders | x | x | x | x |
| synthetic mica substrate | Silica powders | x | x | x | x |
| coated with a metal | Poly(methyl | x | x | x | x |
| compound, in particular | methacrylate) | | | | |
| titanium dioxide | (PMMA) powders | | | | |
| | Elastomeric | x | x | x | x |
| | organopolysiloxane | | | | |
| | powders | | | | |
| | Hydrophobic silica | x | x | x | x |
| | aerogel powders | | | | |
| | Cellulose powders | x | x | x | x |
| | and elastomeric | | | | |
| | organopolysiloxane | | | | |
| | powders | | | | |
| | Poly(methyl | x | x | x | x |
| | methacrylate) | | | | |
| | (PMMA) and | | | | |
| | hydrophobic silica | | | | |
| | aerogel powders | | | | |
| Particles comprising | Cellulose powders | x | x | x | x |
| an alumina substrate | Silica powders | x | x | x | x |
| coated with a metal | Poly(methyl | x | x | x | x |
| compound, in particular | methacrylate) | | | | |
| titanium dioxide | (PMMA) powders | | | | |
| | Elastomeric | x | x | x | x |
| | organopolysiloxane | | | | |
| | powders | | | | |
| | Hydrophobic silica | x | x | x | x |
| | aerogel powders | | | | |
| | Cellulose powders | x | x | x | x |
| | and elastomeric | | | | |
| | organopolysiloxane | | | | |
| | powders | | | | |
| | Poly(methyl | x | x | x | x |
| | methacrylate) | | | | |
| | (PMMA) and | | | | |
| | hydrophobic silica | | | | |
| | aerogel powders | | | | |
| Particles comprising an | Cellulose powders | x | x | x | x |
| alumina substrate coated | Silica powders | x | x | x | x |
| with a metal compound, | Poly(methyl | x | x | x | x |
| in particular titanium | methacrylate) | | | | |
| dioxide and Particles | (PMMA) powders | | | | |
| comprising a synthetic mica | Elastomeric | x | x | x | x |
| substrate coated with a | organopolysiloxane | | | | |
| metal compound, in | powders | | | | |
| particular titanium dioxide | Hydrophobic silica | x | x | x | x |
| | aerogel powders | | | | |
| | Cellulose powders | x | x | x | x |
| | and elastomeric | | | | |
| | organopolysiloxane | | | | |
| | powders | | | | |
| | Poly(methyl | x | x | x | x |
| | methacrylate) | | | | |
| | (PMMA) and | | | | |
| | hydrophobic silica | | | | |
| | aerogel powders | | | | |

According to a particularly preferred embodiment, the cosmetic composition according to the invention comprises the combination with universal optical correction effect consisting of:
(i) at least one soft-focus effect filler as defined above in a content ranging from 1 to 10 wt % based on the total weight of the composition,
(ii) at least one composite pigment as defined above in a content ranging from 1 to 10 wt % based on the total weight of the composition, and
(iii) at least one reflective particle as defined above in a content ranging from 0.1 to 5 wt % based on the total weight of the composition.

According to a preferred embodiment, the cosmetic composition comprises the combination with universal optical correction effect consisting of:
(i) at least one soft-focus effect filler selected from cellulose powders, poly(methyl methacrylate) (PMMA) powders, cross-linked elastomeric organopolysiloxane powders coated with silicone resin, hydrophobic silica aerogel powders, preferably in a total content ranging from 1 to 10 wt % based on the total weight of the composition;
(ii) at least one composite pigment selected from inorganic pigments embedded in a silica matrix, inorganic pigments embedded in a PMMA matrix, preferably in a total content ranging from 1 to 10 wt % based on the total weight of the composition; and
(iii) at least one reflective particle selected from particles comprising a synthetic mica substrate coated with titanium dioxide, particles comprising an alumina substrate coated with titanium dioxide, preferably in a total content ranging from 0.1 to 5 wt % based on the total weight of the composition.

According to a preferred embodiment of the invention, the cosmetic composition comprises the combination with universal optical correction effect consisting of:
(i) a combination of cross-linked elastomeric organopolysiloxane elastomeric powders coated with silicone resin and cellulose powders as soft-focus effect fillers, preferably in a total content ranging from 1 to 10 wt % based on the total weight of the composition;
(ii) inorganic pigments embedded in a silica matrix as composite pigments, preferably in a total content ranging from 1 to 10 wt % based on the total weight of the composition; and
(iii) a combination of particles comprising a synthetic mica substrate coated with titanium dioxide and particles comprising an alumina substrate coated with titanium dioxide as reflective particles, preferably in a total content ranging from 0.1 to 5 wt % based on the total weight of the composition.

According to another preferred embodiment of the invention, the cosmetic composition comprises the combination with universal optical correction effect consisting of:
(i) a combination of poly(methyl methacrylate) (PMMA) powders and hydrophobic silica aerogel powders as soft-focus effect fillers, preferably in a total content ranging from 1 to 10 wt % based on the total weight of the composition;
(ii) inorganic pigments embedded in a PMMA matrix as composite pigments, preferably in a total content ranging from 1 to 10 wt % based on the total weight of the composition; and
(iii) a combination of particles comprising a synthetic mica substrate coated with titanium dioxide and particles comprising an alumina substrate coated with titanium dioxide as reflective particles, preferably in a total content ranging from 0.1 to 5 wt % based on the total weight of the composition.

According to still another preferred embodiment of the invention, the cosmetic composition comprises the combination with universal optical correction effect consisting of:
(i) poly(methyl methacrylate) (PMMA) powders as soft-focus effect fillers, preferably in a total content ranging from 1 to 10 wt % based on the total weight of the composition;
(ii) inorganic pigments embedded in a silica matrix as composite pigments, preferably in a total content ranging from 1 to 10 wt % based on the total weight of the composition; and
(iii) particles comprising an alumina substrate coated with titanium dioxide as reflective particles, preferably in a total content ranging from 0.1 to 5 wt % based on the total weight of the composition.

The cosmetic composition may also comprise other additional particles selected from fillers, pigments, and mixtures thereof, distinct from the soft-focus effect fillers, reflective particles and composite pigments mentioned above, provided that their presence does not have an impact on the desired effect.

According to a particular embodiment, the composition is devoid of additional particles selected from fillers, pigments, and mixtures thereof.

Galenical

The cosmetic composition of the invention is advantageously in the form of an oil-in-water emulsion, a water-in-oil emulsion, a water-in-silicone emulsion, a silicone-in-water emulsion, a multiple emulsion, an aqueous gel.

The composition is preferably in the form of an emulsion containing an aqueous phase and an oil phase.

The aqueous phase generally represents from 1 to 99 wt %, based on the total weight of said composition.

The composition is preferentially intended to be applied to the face and is preferably in the form of an oil-in-water, water-in-oil or water-in-silicone emulsion or an aqueous gel. The composition is for example in the form of a care cream, lotion, serum or fluid for the face, a foundation, or a foundation primer, a “finisher”.

The composition of the invention generally includes, in addition to the aqueous phase, an oil phase as well.

“Oil phase” means an oil or an intermiscible mixture of oils. For the purposes of the invention, “oil” means a fatty substance which is water-insoluble, liquid at 25° C. and 0.1 MPa, and preferably non-volatile having a non-zero vapor pressure, at 25° C. and 0.1 MPa, less than 2.6 Pa, preferably less than 0.13 Pa.

An oil phase according to the invention may comprise hydrocarbon, silicon, fluorinated oils or not, and mixtures thereof.

These oils can be volatile or non-volatile, vegetable, mineral or synthetic.

According to the invention, “volatile oil” means an oil of a volatile nature defined by at least one of the criteria defined below.

The volatility may be defined in the context of the invention, for example a vapor pressure measurable by an empirical method at 25° C., and whose value will be comprised between 0.13 Pa and 40,000 Pa, for example between 1 Pa and 20,000 Pa, between 10 Pa and 8,000 Pa, or between 15 and 150 Pa. The vapor pressure will be measured using one of the most appropriate methods for the compound of interest, which methods appear in the OECD Test No. 104 (2006 version) guidelines. Alternatively, a volatile oil with a boiling temperature at atmospheric pressure below 250° C., preferably below 230° C. and preferably comprised between 150° C. and 220° C., can be selected. Finally, volatile oil can further be defined as an oil with a flash point ranging from 35° C. to 100° C., preferably between 40° C. and 80° C.

According to the invention, “non-volatile oil” means an oil that does not meet the volatility criteria defined above.

According to the invention, “hydrocarbon oil” means an oil containing mainly hydrogen and carbon atoms.

According to the invention, “silicon oil” means an oil containing at least one silicon atom, and in particular at least one Si—O group.

According to the invention, “fluorinated oil” means an oil containing at least one fluorine atom.

As volatile hydrocarbon oils, particular mention may be made of branched C₈-C₁₆ alkanes, branched C₈-C₁₆ esters and mixtures thereof.

As volatile silicon oils, particular mention may be made of linear or cyclic volatile silicon oils and mixtures thereof.

As non-volatile hydrocarbon oils, particular mention may be made of hydrocarbon oils, vegetable hydrocarbon oils, C₁₀-C₄₀ synthetic ethers, C₁₀-C₄₀ synthetic esters, C₁₂-C₂₆ fatty alcohols, C₁₂-C₂₂ higher fatty acids, and mixtures thereof.

As non-volatile silicon oils, particular mention may be made of phenylated silicon oils, non-phenylated silicon oils, and mixtures thereof.

The non-volatile silicon oils that can be used in the composition according to the invention may be non-volatile polydimethylsiloxanes (PDMS), polydimethylsiloxanes containing at least one alkyl or alkoxy group, C₂₋₂₄, hanging and/or at the end of the silicon chain, phenylated silicones such as phenyl trimethicones, phenyl dimethicones, phenyl trimethylsiloxy diphenylsiloxanes, diphenyl dimethicones, diphenyl methyldiphenyl trisiloxanes, 2-phenylethyl trimethylsiloxysilicates.

The volatile oils used in the composition of the invention are preferably volatile silicones or saturated, branched-chain hydrocarbons.

The volatile oil may notably be selected from silicon oils such as dimethicones (polydimethylsiloxanes) whose viscosity ranges from 0.5 to 6 cSt and cyclomethicones.

The volatile oil may be isohexyl neopentanoate or a hydrocarbon such as isododecane, isodecane, isohexadecane, n-dodecane (C₁₂) and n-tetradecane (C₁₄) or the mixture of undecane and tridecane.

Mention may be made, for example, of octamethyl cyclotetrasiloxane, decamethyl cyclopentasiloxane, dodecamethyl cyclohexasiloxane, heptamethyl hexyltrisiloxane, heptamethyl trisiloxane, hexamethyl disiloxane, octamethyl trisiloxane, decamethyl tetrasiloxane, dodecamethyl pentasiloxane, and mixtures thereof.

The oils may be present in the composition of the invention in a content ranging from 1 to 95 wt % based on the total weight of the composition.

The composition of the invention may also comprise any additive commonly used in cosmetics such as UV filters, antioxidants, surfactants, gelling agents, preservatives, film-forming polymers, fragrances, cosmetic active agents, such as for example emollients, moisturizers, vitamins, anti-aging agents, lightening agents, and mixtures thereof.

Another object of the invention is a cosmetic process for the care and/or make-up of keratinous materials, in particular the skin, comprising at least one step consisting in applying to said keratinous material a composition as defined above.

In particular, the cosmetic process of the invention is intended to mask in transparency the imperfections of relief and/or color of the skin, while providing a light effect and adapting to all skin complexions.

The present invention also relates to the combination of at least one soft-focus effect filler, at least one composite pigment, and at least one reflective particle, as defined above, for universal optical correction regardless of complexion.

The present invention will be illustrated by the following non-limiting examples. The % are expressed as wt % of raw material based on the total weight of the composition, unless otherwise specified.

### EXAMPLES

### Example 1: Oil-In-Water Emulsion with Overall Corrective Effect

The following combination is used to obtain a universal optical correction effect:
as soft-focus effect fillers, cross-linked elastomeric organopolysiloxane powders coated with Polyquaternium-7 silicone resin and methylsilanol tri-PEG-8 glyceryl cocoate marketed by Miyoshi Kasei under the name MW-SRP100 and the cellulose powder CELLULOBEADS USF,
as composite pigments spherical silica particles in which inorganic pigments, in particular titanium oxides and dioxide, iron oxides and mixtures thereof, are encapsulated; such as SPHERICA HC RW 30 and SPHERICA HC YW30 and SPHERICA HC BW30 and SPHERICA HC WHITE 30 from JGC,
as reflective particles, particles comprising a synthetic mica substrate coated with titanium dioxide CI77891 under the name Timiron Halo White from MERCK; and
particles comprising an alumina substrate coated with titanium dioxide under the name SpectraFlex Focus White from SUN CHEMICAL.

An oil-in-water emulsion comprising said combination is prepared according to the following protocol:
the aqueous phase (water and glycols) is homogenized under stirring (350 rpm);
gelling agents are added in the phase under stirring (2 h, 1500 rpm)
the aqueous silicone elastomer dispersion is added (1000 rpm, 15 min)
the mixture is neutralized with sodium hydroxide.
the previously homogenized oils are added at 40° C. (1000 rpm for 15 min)
acrylate copolymers (rheological agent) are added 1000 rpm 10 min
the compounds of the combination with optical correction effect are added (1300 rpm for 10 min).

| | |
|---|---|
| WATER | QS 100 |
| BUTYLENE GLYCOL | 5.6% |
| GLYCERIN | 5.0% |
| PENTAERYTHRITYL TETRAISOSTEARATE | 3.0% |
| DIPROPYLENE GLYCOL | 2.0% |
| DIMETHICONE | 1.7% |
| ACRYLATES COPOLYMER | 6.4% |
| PHENOXYETHANOL | 0.7% |
| SODIUM CARBOXYMETHYL DEXTRAN | 0.5% |
| POLYSILICONE-11 | 0.14% |
| SODIUM HYDROXIDE | 0.08% |
| TETRASODIUM EDTA | 0.05% |
| CROSS-LINKED ELASTOMERIC ORGANOPOLYSILOXANE POWDERS | 3.0% |
| COATED WITH POLYQUATERNIUM-7 SILICONE RESIN AND | |
| METHYLSILANOL TRI-PEG-8 GLYCERYL COCOATE MARKETED BY | |
| MIYOSHI KASEI UNDER THE NAME MW-SRP100 (SOFT-FOCUS | |
| EFFECT FILLER) | |
| CELLULOSE POWDER - CELLULOBEADS USF (SOFT-FOCUS | 1.0% |
| EFFECT FILLER) | |
| SPHERICAL SILICA PARTICLES IN WHICH RED IRON OXIDES ARE | 0.37% |
| EMBEDDED - SPHERICA HC RW 30 FROM JGC (COMPOSITE | |
| PIGMENTS) | |
| SPHERICAL SILICA PARTICLES IN WHICH YELLOW IRON OXIDES | 0.59% |
| ARE EMBEDDED - SPHERICA HC YW30 FROM JGC (COMPOSITE | |
| PIGMENTS) | |
| SPHERICAL SILICA PARTICLES IN WHICH BROWN IRON OXIDES | 0.15% |
| ARE EMBEDDED - SPHERICA HC BW30 FROM JGC (COMPOSITE | |
| PIGMENTS) | |
| SPHERICAL SILICA PARTICLES IN WHICH TITANIUM DIOXIDE IS | 0.1% |
| EMBEDDED - SPHERICA HC WHITE 30 FROM JGC (COMPOSITE | |
| PIGMENTS) | |
| PARTICLES COMPRISING A SYNTHETIC MICA SUBSTRATE COATED | 0.2% |
| WITH TITANIUM DIOXIDE CI77891 UNDER THE NAME TIMIRON HALO | |
| WHITE FROM MERCK (REFLECTIVE PARTICLES) | |
| PARTICLES COMPRISING AN ALUMINA SUBSTRATE COATED WITH | 0.5% |
| TITANIUM DIOXIDE UNDER THE NAME SPECTRAFLEX FOCUS | |
| WHITE FROM SUN CHEMICAL (REFLECTIVE PARTICLES) | |

This oil-in-water emulsion has a ΔE*₀₀ of less than 10 in accordance with the invention, comprised between 1 (for lighter skin) and 9 (for darker skin) measured according to the above-mentioned protocol.

Applied to the skin of the face, it provides an overall corrective effect: blurred imperfections, even complexion and luminous finish.

### Example 2: Water-In-Oil Emulsion with Overall Corrective Effect

The following combination is used to obtain a universal optical correction effect:
As soft-focus effect fillers, the PMMA powders with the INCI name “methyl methacrylate crosspolymer” MAKIBEADS 150 from Daito Kasei and the hydrophobic silica aerogel powder VM-2270 Aerogel from Dow Corning;
As composite pigments, hemispherical particles of poly(methyl methacrylate) (PMMA) coated with inorganic pigments marketed under the name 3D TECH PW OTS IOR-30 and 3D TECH PW OTS IOY-30 and 3D TECH PW OTS IOB-30 and 3D TECH PW OTS TIO-30 marketed by DAITO;
As reflective particles, the particles comprising a synthetic mica substrate coated with CI77891 titanium dioxide under the name Timiron Halo White from MERCK; and particles comprising an alumina substrate coated with titanium dioxide under the name SpectraFlex Focus White from SUN CHEMICAL.

A water-in-oil emulsion comprising said combination is prepared according to the following protocol:
the oil phase comprising lipophilic oils and gelling agents is mixed with an Ultra Turrax mixer,
the combination with optical correction effect is added at 500 rpm for 10 min,
the aqueous phase is solubilized with a Rayneri mixer, the hydrophilic gelling agent pre-mixed in glycerol is added,
the aqueous phase is gradually added to the oil phase under mixing with a Rayneri mixer, gradually increasing the stirring speed to 1100 rpm, at which speed stirring continues for 15 min,
preservatives are added.

| | |
|---|---|
| WATER | QS 100% |
| CYCLOPENTASILOXANE | 29.5% |
| GLYCERIN | 8.0% |
| BUTYLENE GLYCOL DICAPRYLATE/DICAPRATE | 6.5% |
| ISODODECANE | 3.9% |
| PENTYLENE GLYCOL | 3.0% |
| DIMETHICONE | 2.0% |
| PEG-10 DIMETHICONE | 1.4% |
| SESQUIOLEATE SORBITAN | 1.0% |
| DISTEARDIMONIUM HECTORITE | 1.0% |
| PHENOXYETHANOL | 0.9% |
| ACRYLATES/DIMETHICONE COPOLYMER | 0.6% |
| XANTHAN GUM | 0.3% |
| PROPYLENE CARBONATE | 0.14% |
| PMMA POWDERS UNDER THE INCI NAME “METHYL | 3.0% |
| METHACRYLATE CROSSPOLYMER” MAKIBEADS 150 FROM DAITO | |
| KASEI (SOFT-FOCUS EFFECT FILLER) | |
| HYDROPHOBIC SILICA AEROGEL POWDER VM-2270 AEROGEL | 1.0% |
| FROM DOW CORNING (SOFT-FOCUS EFFECT FILLER) | |
| HEMISPHERICAL POLY(METHYL METHACRYLATE) (PMMA) | 0.38% |
| PARTICLES COATED WITH RED IRON OXIDES - 3D TECH PW OTS | |
| IOR-30 FROM DAITO (COMPOSITE PIGMENTS) | |
| HEMISPHERICAL POLY(METHYL METHACRYLATE) (PMMA) | 0.8% |
| PARTICLES COATED WITH YELLOW IRON OXIDES - 3D TECH PW | |
| OTS IOY-30 FROM DAITO (COMPOSITE PIGMENTS) | |
| HEMISPHERICAL POLY(METHYL METHACRYLATE) (PMMA) | 0.16% |
| PARTICLES COATED WITH BROWN IRON OXIDES - 3D TECH PW | |
| OTS IOB-30 FROM DAITO (COMPOSITE PIGMENTS) | |
| HEMISPHERICAL POLY(METHYL METHACRYLATE) (PMMA) | 0.2% |
| PARTICLES COATED WITH TITANIUM DIOXIDE - 3D TECH PW OTS | |
| TIO-30 FROM DAITO (COMPOSITE PIGMENTS) | |
| PARTICLES COMPRISING A SYNTHETIC MICA SUBSTRATE | 0.2% |
| COATED WITH TITANIUM DIOXIDE CI77891 UNDER THE NAME | |
| TIMIRON HALO WHITE FROM MERCK (REFLECTIVE PARTICLES) | |
| PARTICLES COMPRISING AN ALUMINA SUBSTRATE COATED | 0.5% |
| WITH TITANIUM DIOXIDE UNDER THE NAME SPECTRAFLEX | |
| FOCUS WHITE FROM SUN CHEMICAL (REFLECTIVE PARTICLES) | |

This water-in-oil emulsion has a ΔE*₀₀ of less than 20 or even less than 10, according to the invention, since it is between 2 (for the lightest skin) and 9 (for the darkest skin) measured according to the above-mentioned protocol.

Applied to the skin of the face, it provides an overall corrective effect: imperfections are blurred, the complexion is even and with a luminous finish.

### Example 3: Aqueous Gel with Overall Corrective Effect

The following combination is used to obtain a universal optical correction effect:
As soft-focus effect fillers, PMMA powders such as the powder with INCI name “methyl methacrylate crosspolymer” from Sunjin under the name SUNPMMA-X,
As composite pigments, spherical silica particles in which are encapsulated respectively red, yellow and brown iron oxides and titanium dioxide marketed under the names SPHERICA HC RW 30 and SPHERICA HC YW30 and SPHERICA HC BW30 and SPHERICA HC WHITE 30 from JGC, and
As reflective particles, the particle comprising an alumina substrate coated with titanium dioxide under the name SpectraFlex Focus White from SUN CHEMICAL.

An aqueous gel comprising said combination is prepared according to the following protocol:
the aqueous phase is heated to 80° C.,
the gelling agent is introduced under mixing with a Rayneri mixer,
the composite pigments are introduced under stirring,
the preservatives are added,
the aqueous phase is cooled to 60° C.
the oil phase is heated to 60° C. and then introduced into the aqueous phase under rapid stirring (1000 rpm)
the rest of the combination with optical correction effect is introduced: soft-focus effect fillers and reflective particles.

| | |
|---|---|
| WATER | QS 100% |
| ISOSTEARYL ISOSTEARATE | 12.5% |
| DIPROPYLENE GLYCOL | 7.0% |
| GLYCERIN | 3.5% |
| PEG/PPG/POLYBUTYLENE GLYCOL-8/5/3 GLYCERIN | 3.0% |
| BUTYLENE GLYCOL | 3.0% |
| ACRYLATES COPOLYMER | 1.3% |
| PHENOXYETHANOL | 0.5% |
| PEG-240/HDI COPOLYMER BIS-DECYLTETRADECETH-20 ETHER | 0.5% |
| CAPRYLYL GLYCOL | 0.3% |
| HYDROGENATED POLYISOBUTENE | 0.21% |
| PMMA POWDERS SUCH AS THE POWDER WITH INCI NAME | 1.0% |
| “METHYL METHACRYLATE CROSSPOLYMER” FROM SUNJIN UNDER | |
| THE NAME SUNPMMA-X (SOFT-FOCUS EFFECT FILLER) | |
| SPHERICAL SILICA PARTICLES IN WHICH RED IRON OXIDES ARE | 0.52% |
| EMBEDDED - SPHERICA HC RW 30 FROM JGC (COMPOSITE | |
| PIGMENTS) | |
| SPHERICAL SILICA PARTICLES IN WHICH YELLOW IRON OXIDES | 2.4% |
| ARE EMBEDDED - SPHERICA HC YW30 FROM JGC (COMPOSITE | |
| PIGMENTS) | |
| SPHERICAL SILICA PARTICLES IN WHICH BROWN IRON OXIDES | 0.015% |
| ARE EMBEDDED - SPHERICA HC BW30 FROM JGC (COMPOSITE | |
| PIGMENTS) | |
| SPHERICAL SILICA PARTICLES IN WHICH TITANIUM DIOXIDE IS | 6.0% |
| EMBEDDED - SPHERICA HC WHITE 30 FROM JGC (COMPOSITE | |
| PIGMENTS) | |
| PARTICLES COMPRISING AN ALUMINA SUBSTRATE COATED WITH | 2.0% |
| TITANIUM DIOXIDE UNDER THE NAME SPECTRAFLEX FOCUS | |
| WHITE FROM SUN CHEMICAL (REFLECTIVE PARTICLES). | |

This aqueous gel has a ΔE*₀₀ of less than 20 in accordance with the invention, comprised between 1 (for lighter skin) and 19 (for darker skin) measured according to the above-mentioned protocol.

Applied to the skin of the face, it provides an overall corrective effect: imperfections are blurred, the complexion is even and with a luminous finish.

1-10. (canceled)

11. Cosmetic composition in the form of a water-in-silicone emulsion, comprising an aqueous phase, a silicone phase, and one combination with optical correction effect adapted to all skin complexions, comprising
(i) at least one soft-focus effect filler,
(ii) at least one composite pigment, and
(iii) at least one reflective particle,
wherein:
the at least one soft-focus effect filler is a poly(methyl methacrylate) (PMMA) powder,
the at least one composite pigment comprises iron oxide(s) embedded in a PMMA substrate or matrix, and
the at least one reflective particle is a particle with a natural or synthetic mica or alumina substrate coated at least partially with at least one layer of titanium dioxide,
wherein said cosmetic composition blurs and colors the skin, and
wherein said cosmetic composition achieves an optical correction effect adapted to all skin complexions and achieves a color difference (ΔE*₀₀) between bare skin and the same skin onto which said cosmetic composition is applied ranges from 2 to 9 as defined in Standard 11664-6:2014 of the ISO/CIE.

12. The cosmetic composition according to claim 11, wherein said silicone phase comprises volatile silicon oils and non-volatile polydimethylsiloxanes (PDMSs).

13. The cosmetic composition according to claim 11, wherein silicone compounds in said silicone phase represent from 30% to 50% by weight of the total weight of the composition.

14. The cosmetic composition according to claim 11, wherein said reflective particle(s) has/have a D90 less than or equal to 40 μm.

15. The cosmetic composition according to claim 11, wherein said reflective particle(s) is/are particle(s) with alumina or synthetic mica substrate coated with inorganic and/or organic pigments.

16. A cosmetic method for the care and/or make-up of keratinous materials, comprising at least one step of applying onto skin in need thereof of the cosmetic composition according to claim 11.

17. The cosmetic method according to claim 16, wherein imperfections of relief and/or color of said skin are masked in transparency while providing a light effect and adapted to all skin complexions.

18. A cosmetic care kit for blurring and coloring the skin, comprising
(a) a first composition comprising an aqueous phase, and
(b) at least one soft-focus effect filler, at least one composite pigment, and at least one reflective particle, packaged in one or more compositions distinct from the first composition, for the extemporaneous preparation of a cosmetic composition as defined in claim 11.
